(19) United States
(12) Patent Application Publication
(10) Pub. No.: US 2007/0004971 A1
(43) Pub. Date: Jan. 4, 2007

(54) CAREGIVER COMMUNICATION SYSTEM FOR A HOME ENVIRONMENT

(75) Inventors: Carl W. Riley, Milan, IN (US); Keith A. Huster, Sunman, IN (US); Williams F. Collins JR., Columbus, IN (US); Patricia A. Glidewell, Apex, NC (US); James M. Allen, Batesville, IN (US)

Correspondence Address: BARNES & THORNBURG LLP, 11 SOUTH MERIDIAN, INDIANAPOLIS, IN, 46204, US

(73) Assignee: HILL-ROM SERVICES, INC., Wilmington, DE (US)

(21) Appl. No.: 11/419,803

(22) Filed: May 23, 2006

Related U.S. Application Data
(60) Provisional application No. 60/685,639, filed on May 27, 2005.

(51) Int. Cl. A61B 5/00

(52) U.S. Cl. 600/300

(57) ABSTRACT

A caregiver communication system for a home environment provides remote alert condition setting and monitoring of a patient and patient support equipment. A patient communication portal located in the patient home environment may use an existing telecommunications infrastructure to communicate with a monitoring communication portal located in a geographically distant location, for example, in a hospital staffed with caregivers responsible for monitoring the in-home patient and equipment. The monitoring communication portal can be interfaced with existing nurse communication systems and/or a hospital network infrastructure to link the communication system with caregiver communication devices such as phones, intercoms, pagers, wireless handsets, badges, and the like.

### CROSS-REFERENCE TO RELATED APPLICATIONS

This application claims the benefit under 35 U.S.C. § 119(e) of U.S. Provisional Application Ser. No. 60/685,639, filed May 27, 2005, which is expressly incorporated by reference herein.

### BACKGROUND

The present invention relates to a healthcare communication system, and particularly, to a communication system having a bed and device status system for providing patient monitoring by caregivers.

Hospital nurse communication systems provide instant communication between patients, caregivers, and dispatchers and also provide monitoring of patient support equipment such as a hospital bed, vital signs, and various patient care devices from a nurse station or other central location. In-home patient care arrangements provide special challenges for caregivers responsible for the patient's care. Caregivers often only make periodic visits, so when a health or equipment problem arises patients must contact the caregiver by telephone to discuss such problems. Such problems may not always be resolved over the telephone and may have to wait for the next scheduled visit, or the caregiver must make an unscheduled visit to the patient's home. Additionally, patient health or equipment problems may go unnoticed until the next in-home visit by the caregiver.

### SUMMARY

The present invention may comprise one or more of the features recited in the attached claims, and/or one or more of the following features and combinations thereof. A patient monitoring system may comprise a patient support system for a home environment, a sensor system configured to generate a first signal relating to at least one parameter of the patient support system, a first communication portal coupled to the sensor system by a first datalink, and a second communication portal positioned geographically distant from the first communication portal. The first communication portal is configured to receive the first signal from the sensor system and the second communication portal which is coupled to the first communication portal by a second datalink system is configured to receive the first signal from the first communication portal. A monitoring device is coupled to the second communication portal by a third datalink system and is configured to receive and process the first signal from the second communication portal. A caregiver interface configured to provide indications relating to the first signal may be coupled to the monitoring device. A caregiver interface configured to provide monitoring of the first signal may be coupled to the monitoring device, for example by a third communication portal. A patient interface coupled to the first communication portal by the first datalink may be configured to provide voice communication between a patient and caregiver.

The patient support system may include at least one of a patient support surface, for example a hospital bed, and a patient care device, for example medical equipment such as an IV pump. A forth communication portal coupling the first communication device and the patient care device may be configured to receive a second signal from the patient care device and transmit the second signal to the first communication device. The second signal relates to at least one parameter of the patient care device. The first datalink may also include at least one communication controller adapted to combine the first signal received from the sensor system and the second signal received from the forth communication portal for transmission to the first communication portal. The at least one communication controller may also be configured to separate a plurality of signals received from the first communication portal for transmission to one of the sensor system and the forth communication portal. The at least one communication controller coupled the first communication portal and the forth communication portal.

The first communication portal may be configured to periodically transmit the first and second signals to the monitoring device and may be configured to transmit a third signal to the monitoring device upon a parameter associated with at least one of the sensor system and the patient care device violating a predefined condition. The predefined condition may be transmitted from the monitoring device to the first communication portal. The monitoring device may include a component of a nurse call system. The second datalink may include at least one of a wireless network, internet, and telecommunications network. The third datalink may include a component of a hospital network infrastructure.

A patient monitoring system for a patient support system may comprise a patient support system sensor configured to generate signals relating to a status of the patient support system, a first communication portal coupled to the patient system sensor and configured to receive and transmit the signals, a second communication portal located geographically distant from the first communication portal, and a monitoring device coupled to the second communication portal and configured to receive the signals from the second communication portal and provide an alert to a caregiver upon the signals violating one of a plurality of conditions. The second communication portal may be configured to be coupled to the first communication portal by a datalink and to receive the signals from the first communication portal and transmit the signals to the monitoring device. The datalink may include at least one of a wireless network, internet, and a telecommunications network. The second communication portal may include a component of a hospital network infrastructure.

The patient support system sensor may be associated with, and monitor a parameter relating to a status of, at least one of a patient support surface, a patient monitoring device, an IV pump, a sequential compression device, a pulmonary percussion therapy device, an environmental monitoring device, and an oxygen concentrator. Alternatively, or additionally, the patient support system sensor may monitor at least one of patient blood pressure, temperature, respiration rate, blood oxygen saturation, and heart rate. Alternatively, or additionally, the patient support system sensor may monitor a parameter relating to at least one of patient position, patient weight, a performance value for a component of the patient support surface, the position of a component of the patient support surface, pressure relief component of the patient support surface, and a percussion therapy component of the patient support surface.

The patient monitoring system may further comprise a caregiver interface coupled to the monitoring device and configured to allow a caregiver to set the plurality of predefined conditions. The monitoring system may also be configured to store the plurality of predefined conditions and to provide the alert based on comparing the received signals to the plurality of predefined conditions. The predefined conditions may relate to a patient standard of care. The caregiver interface may be configured to annunciate the alert. The patient monitoring system may further comprise a patient interface coupled to the first communication portal and configured to provide voice communication between a patient and a caregiver. The monitoring device may include a component of a nurse call system. The first communication portal may be configured to receive and store the plurality of predefined conditions and further configured to initiate a communication connection through the datalink and the second communication portal to provide an alert signal to the monitoring device upon signals violating at least one of the plurality of predefined conditions. The first communication portal may also be configured to periodically initiate a communication connection through the datalink and the second communication portal and further configured to provide the signals relating to a status of the patient support system upon initiation of the communication connection.

The patient monitoring system for an in-home patient support system may comprise a sensor system configured to generate signals relating to at least one parameter of the patient support system, a first communication portal coupled to the sensor system by a first datalink, a second communication portal located geographically distant from the first communication portal and coupled to the first communication portal by a second datalink, and a monitoring device coupled to the second communication portal by a third datalink, the monitoring device configured to store conditions relating to the signals. The first communication portal, the second communication portal, and the monitoring device may be configured to transmit and receive data relating to the signals and the conditions. The second datalink may include at least one of a wireless network, internet, and telecommunications network. The third datalink may include a component of a hospital network infrastructure. The monitoring device may include a component of a nurse call system.

The monitoring device may further include a processor and software enabling the monitoring device to provide selection of conditions relating to the at least one parameter of the patient support system, receive the data relating to the signals and the conditions, compare the conditions to the received data, and provide a caregiver alert upon the received data satisfying the conditions. The software may further enable the monitoring device to periodically command the first communication device to send the data. The software may further enable the monitoring device to transmit conditions to the first communication device.

The first communication device may include a processor and software enabling the first communication device to receive conditions relating to the at least one parameter of the patient support system, receive signals relating to the at least one parameter of the patient support system, and selectively transmit data to the monitoring device based on a function of the monitoring and alert conditions and signals. The data may be transmitted at least periodically. Alternatively, or additionally, the data may be transmitted at least when any of the signals satisfy the associated conditions. The first communication device may also include a second communication device, the first communication device being associated with a patient support surface and the second communication device being associated with a patient care device.

The patient monitoring system may further include a patient interface coupled to the first communication device, and a caregiver interface coupled to the second communication device, the patient interface and caregiver interface being configured to provide voice communication between a patient and a caregiver.

Additional features of the disclosure will become apparent to those skilled in the art upon consideration of the following detailed description of the illustrative embodiment.

### BRIEF DESCRIPTION OF THE DRAWINGS

The detailed description particularly refers to the accompanying figures, in which:

FIG. 1 is a map showing how to layout FIGS. 1A and 1B to form a block diagram of an illustrative embodiment of a caregiver communication system for a home environment in which medical equipment at a patient's home communicates with monitoring devices at a monitoring facility that is geographically distant from the patient's home;

FIG. 2 is a flow chart illustrating exemplary software associated with the in-home portion of the patient monitoring system of FIG. 1; and

FIG. 3 is a flow chart illustrating exemplary software associated with the monitoring facility portion of the patient monitoring system of FIG. 1.

### DESCRIPTION OF THE ILLUSTRATIVE EMBODIMENTS

For the purposes of promoting and understanding the principals of the invention, reference will now be made to one or more illustrative embodiments illustrated in the drawings and specific language will be used to describe the same.

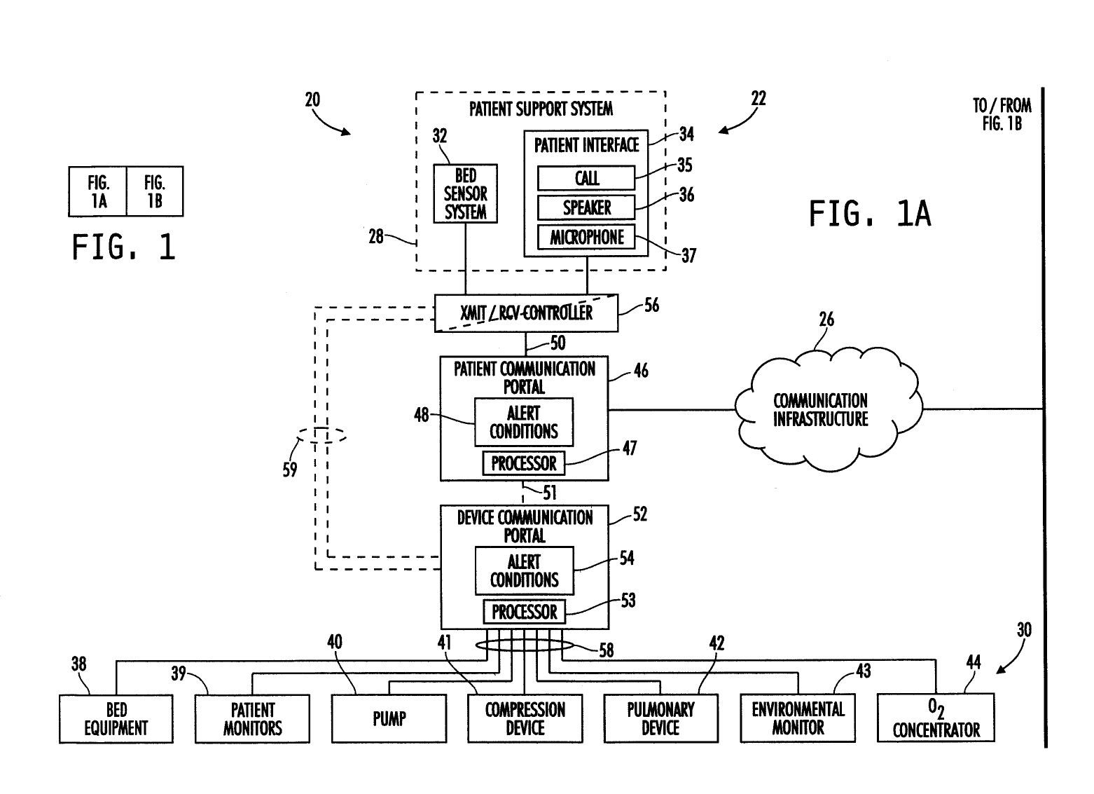

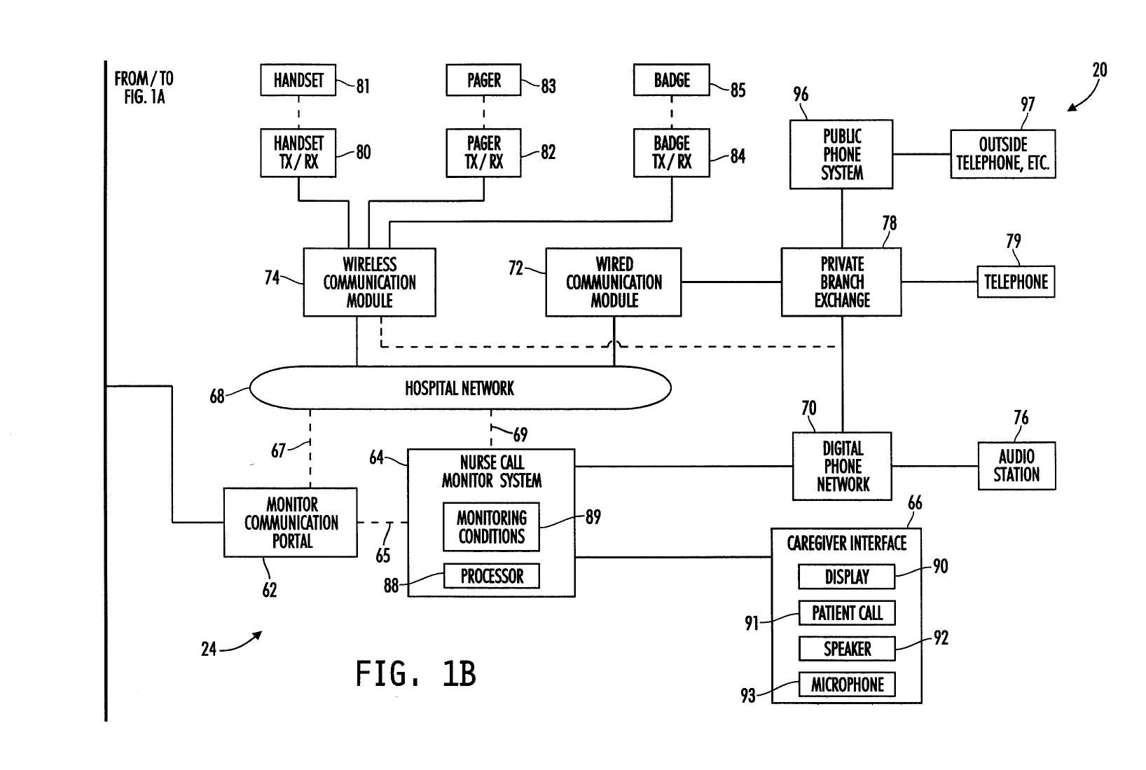

FIGS. 1A and 1B depict an illustrative embodiment of a caregiver communication system 20 for monitoring of a patient and equipment in a home environment. The system 20 generally comprises an in-home portion 22 (FIG. 1A) and a monitoring facility portion 24 (FIG. 1B). The in-home portion 22 is located in a residence or other non-healthcare facility location. The monitoring facility portion 24 is located geographically distant from the in-home portion 22, for example at a hospital or other facility not connected to the home environment and staffed by personnel responsible for the care and/or monitoring of the in-home patient. For example, the personnel may be caregivers, or other personnel responsible for monitoring of the patient and in-home equipment and/or for contacting the caregiver or other designated persons when indicated, and may be located in a facility located across the city or across the country from the patient's home environment.

The in-home portion 22 and the monitoring facility portion 24 may be coupled by a communication infrastructure 26. For the purposes of this disclosure, coupled means directly or indirectly connected so as to provide data transmission and includes wire or wireless connections. The communication infrastructure 26 may be an existing infrastructure such as a wireless network, internet, or a telecommunications network, for example a public switched telephone network, or may be a dedicated infrastructure such as a wired or wireless network, or other dedicated communications link.

The caregiver communication system 20 provides functionality for a patient in a home environment similar to that provided by an in-hospital nurse communication system, for example, the COMLinx™ System, available from Hill-Rom Company, Inc. of Batesville, Ind. For example, the caregiver communication system 20 provides voice communication between the patient and caregiver, and provides patient monitoring information, bed status information, patient care device information, and other healthcare equipment status information to a geographically distant caregiver. Additionally, the caregiver communication system 20 can be linked with wired and wireless communication devices such as telephones, cell phones, and pagers, and can provide predefined alert indication, including for example annunciation, and pre-programmed audio and/or text messaging. For the purpose of this disclosure, a “home environment” includes any non-healthcare facility setting.

The system 20 provides monitoring of the patient and of a patient support system 28 in the home environment. For example, the in-home portion 22 may include a patient support system 28. The patient support system 28 may be, for example, a hospital-type bed or other arrangement having a support surface, and having associated controls as well as accessories carried by the bed, for example, therapeutic accessories. In the illustrative example, system 28 includes a bed sensor system 32, a patient communication interface 34, and one or more patient care devices 30.

The patient interface 34 may be, for example, a pendant type device associated with a hospital bed and may include a nurse call switch 35, an audio speaker 36, and a microphone 37, for supporting voice communication with a caregiver. Alternatively, or additionally, interface 34 may be included as part of a siderail of the hospital bed. The bed sensor system 32 provides monitoring of parameters, including the status, of components and accessories associated with hospital beds. For example, the bed sensor system 32 may provide data signals relating to bed articulation and other controls, and accessories for example, bed height, mattress functions or positioning, head elevation, foot elevation, siderail position, treatment mattress status, patient positioning, patient weight, a performance value for a component of the patient support system, the position of a component of the patient support surface, pressure relief component of the patient support surface, and a percussion therapy of the patient support surface.

The patient care devices 30 that are in communication with the caregiver communication system 20 may include the equipment associated with the patient support system 28, for example wound or other bed accessory equipment 38 and other healthcare devices for supporting and/or monitoring patient status. For example, patient care devices 30 may include, but are not limited to, patient vitals monitor 39, IV pump 40, sequential compression device 41, pulmonary percussion device 42, room environmental monitor 43, and oxygen concentrator 44. Bed equipment 38 may include, for example, a specialty mattress providing pressure relief, rotational therapy, and/or percussion therapy. Patient monitor 39 may include, for example, devices to monitor blood pressure, temperature, respiration rate, blood oxygen saturation, heart rate, and other patient vitals and parameters. Room environmental monitor 43 may include devices to monitor temperature, humidity, light intensity, sound level, air quality, and the like.

The bed sensor system 32 and the patient care devices 30 may be equipped to provide data signals relating to a parameter, i.e. a status or other condition, of the above-listed aspects of the patient and patient support equipment, or of other characteristics and equipment utilized for patient care and monitoring. For example, the signals may be used to communicate a wide range of parameters relating to the status or condition, including, for example, on, off, fault, a range of values, position, temperature, usage counters, usage timers, a warning or alarm, supply voltages, motor or other device current or power draw, or fluid levels.

Components for receiving signals from the bed sensor system 32 and patient care devices 30 may include a patient communication portal 46, a device communication portal 52, and a transmit/receiver controller 56. For this disclosure, a portal is understood to include a device for providing a gateway or access to a communication connection or datalink, and for receiving and transmitting signals across that connection or datalink. A portal may also include a device for signal protocol conversation and may include devices for other signal processing. The patient communication portal 46 is configured to receive data signals from the bed sensor system 32 and receive and send communication signals associated with the patient interface 34. The patient communication portal 46 may also be configured to transmit data signals to the bed sensor system 32. For example, portal 46 may include analog or digital I/O ports, for example serial data ports for communicating with the bed sensor system 32 and the patient interface 34. The I/O ports may also be wireless, for example, radio frequency or infrared ports. The datalink 50 coupling the portal 46 with the bed sensor system 32 and the patient interface 34 may include wired or wireless data connections.

The patient communication portal 46, device communication portal 52, and controller 56 may be integrated with the patient support system 28 or may be separate, stand alone devices in communication with the patient support system 28. The patient communication portal 46 may include a processor 47, memory 48, and associated software for processing and transmitting and/or receiving data signals associated with the bed sensor system 32 and the patient interface 34. For example, the patient communication portal 46 may utilize monitored parameters and alert conditions for processing the data signals received from the bed sensor system 32. The monitored parameters may be the status, condition, or other characteristics associated with the patient support system 28 and associated devices which have been selected for remote monitoring. Such parameters may be transmitted to the monitoring facility portion 24 in the form of signals, for example processed or unprocessed analog or digital signals. The signals associated with the monitored parameters may therefore be processed by either the in-home portion 22 or the monitoring facility portion 24 of the caregiver communication system 20, or by a combination of both the in-home portion 22 and the monitoring facility portion 24.

Alert conditions may include a predefined condition, for example a threshold, range, combination of thresholds and/or ranges, function, and/or Boolean expression, associated with the monitored parameters. Upon being violated, the alert conditions may initiate an action, for example, a transmission of the alert condition, for example, an immediate transmission of a signal indicating that a violation of an alert condition has been determined by the processor 47.

The patient communication portal 46 may transmit the data signals received from the bed sensor system 32, or may transmit other signals that are a function of the data signals received from the bed sensor system 32, for example, a function of the received data signals, including the monitored parameters and alert conditions. Such functions may include Boolean logic function such as an AND function in which two or more conditions must be met by data signals and/or monitored parameters before an alert condition is considered to exist. Software executed by the processor 47 may also provide transmission through the communication infrastructure 26 of the signals and receiving and processing of data signals received from the communication infrastructure 26, for example, receiving and storing parameters conditions and alert conditions. For example, patient communication portal 46 may initiate transmission periodically on a preselected schedule or interval, or upon detection of an alert condition. The datalink 50 coupling the patient communication portal 46 with the bed sensor system 32 and the patient interface 34 may also include a transmitter/receiver controller 56.

The device communication portal 52 is configured to receive signals from the various patient care devices 30. For example, portal 52 may include analog or digital I/O ports, for example serial data ports for communicating with the patient care devices 30. The I/O ports, may also be wireless, for example, radiofrequency or infrared ports. Therefore, datalinks 58 coupling the device communication portal 52 with the patient care devices 30 may include wired or wireless datalinks.

The device communication portal 52 may also include a processor 53, memory 54, and associated software for processing and transmitting the signals received from the patient care devices. For example, the device communication portal 52 may utilize monitored parameters and alert conditions for processing the data signals. The monitored parameters may be the status, condition, or other characteristics associated with the patient care devices 30 which have been selected to be transmitted for remote monitoring. Alert conditions may be predefined conditions associated with the signals or monitored parameters that, upon being violated, initiate a transmission of the alert condition, for example, an immediate transmission of a signal indicating that an alert condition has been determined by the processor 53. The data communication portal 52 may transmit the data signals received from the patient care devices 30, or may transmit other signals that are a function of the data signals received from the patient care devices 30, including the monitoring parameters and alert conditions.

The device communication portal 52 may be directly coupled with the patient communication portal 46 by datalink 51, or indirectly by datalink 59 and the transmitter/receiver controller 56. Datalinks 51 and 59 may be wired or wireless datalinks. The transmitter/receiver controller 56 may comprise a single multiplexing communication controller, or two controllers, one for combining signals received from the device communication portal 52 and the patient support system 28 for transmission over datalink 50 to the patient communication portal 46, and a transmitter controller for separating the data signals received from the patient communication portal 46 for transmission to either the patient support system 28 or the device communication portal 52.

Any one of or all of the transmitter/receiver controller 56, the patient communication portal 46, and the device communication portal 52 may be incorporated into the patient support system 28, for example, as part of the electronic control package of the bed or related equipment. The communication portal 46 may be connected to a telephone outlet, internet connection, or another communication connection, wired or wireless, located in the patient's home environment. In some embodiments, the bed accessory equipment 38 is integrated with the system 28 and communicates with the portal 46 without first communicating with the portal 52. In some embodiments, a single communication portal incorporates the above features of both the patient communication portal 46 and the device communication portal 52. In yet further embodiments, the device communication portal 52 does not store or process alert conditions but transmits the selected parameters of patient care devices 30 in the form of signals for processing by the stored monitored parameters and alert conditions of the patient communication portal 46.

The healthcare facility portion 24 of the caregiver communication system 20, shown in FIG. 1B, includes a monitor communication portal 62 configured to provide communication through the communication infrastructure 26 with the patient communication portal 46, and with a monitoring system, for example, an existing nurse call system that is part of a hospital network infrastructure. The nurse call system may include, for example, a caregiver interface 66 and a nurse call monitor system 64 that are coupled to the monitor communication portal 62 by a datalink 65 or, by datalinks 67 and 69 and a hospital network infrastructure 68. The nurse call system may also include connectivity with other communication devices, either directly with nurse call monitor system 64 or through the hospital network 68. Other communication devices may include other communication portals and datalinks, wired or wireless, for example a digital phone network 70, wired communication module 72, wireless communication module 74, private branch exchange 78, and public switched telephone network 96.

The digital phone network 70 may provide connectivity with audio station 76, for example, caregiver communication stations located at convenient locations throughout a facility, for example, in patient rooms, store rooms, break rooms, and other places caregivers may be located. The wired communication module 72 may provide connectivity through the private branch exchange 78 with facility telephones 79, outside telephones 97, or other wired communication devices known in the art.

The wireless communication module 74 may be used to provide connectivity with wireless communication devices know in the art. A handset transmitter and receiver unit 80 provides wireless connectivity with communication handsets 81, for example, SpectraLink wireless telephones available from SpectraLink Corporation of Boulder, Colo. and/or Ascom handsets available from Ascom Ltd. of Beme, Switzerland. A pager transmitter and receiver unit 82 provides wireless connectivity with pagers 83. A badge transmitter and receiver unit 84 provides wireless connectivity with wireless badges 85, for example, Vocera™ wearable communication devices, available from Vocera Communications, Inc. of Cupertino, Calif. The modules 72, 74 may comprise one or more computer devices such as one or more network servers.

The nurse call monitor system 64 may include a processor 88 and memory 89 for storing data and executing software for processing data signals. The processor 88 and memory 89 may be included in one or more nurse call servers of the system 64. The monitor system 64 receives data signals that are periodically transmitted from the patient communication portal 46, and monitor system 64 may also request transmission of data signals from patient communication portal 46 on a periodic basis or upon data signals having not been received for a predefined period of time. Selection of monitored parameters and alert conditions for caregiver communication system 20 are received and stored by monitor system 64, for example, received from the caregiver interface 66 and stored by the memory 89.

Caregiver interface 66 may include a personal computer, or other terminal including a display and keyboard 90, a patient call selection 91, an audio speaker 92, and a microphone 93. The patient call selection 91 may be, for example, a keyboard or on screen computer mouse or other pointing device selectable feature that is operable to initiate or acknowledge voice communication with the patient. The caregiver interface 66 may be used by a caregiver to define monitored parameters and alert conditions and may be associated with the devices of patient support system 32 and patient care devices 30, to store the selected parameters and conditions in the monitor system 64. The caregiver interface 66 may be also used to transmit the monitored parameters and conditions to the patient communication portal 46 and the device communication portal 52, to monitor both monitored parameters and alert conditions, and to initiate or respond to a request for voice communications with a patient through the patient interface 34. The caregiver interface 66 may also be used by a caregiver to specify the interval of time or other conditions for which the patient communication portal 46 will initiate communication with the monitoring device 64 and transmit signals relating to the monitored parameters and alert conditions.

The monitor system 64 may also provide for selection and storage of predefined protocols, for example a patient standard of care, to which monitored parameters and alert conditions may relate, and response protocols in the event of an alert condition being violated. For example, a response protocol may include contacting particular caregivers, family members of patients, or others based on a particular alert condition, as well as transmitting a prerecorded text or audio message, or escalating the alert to a second caregiver in the event that the alert is not acknowledged by a first. The contact message may be automatically initiated and delivered via the caregiver interface 66 as well as the other above-described communication devices, 79, 81, 83, 85, and 97 and other communication devices know in the art. Additionally, the communication devices may be used to select and define the above-described parameters and alert conditions, rather than using the caregiver interface 66.

The healthcare facility portion 24 of the caregiver communication system 20 may be located at hospital, an independent monitor-for-fee based service location, or any other location that is remote from, but provides communication connectivity with the caregiver.

A caregiver can specify alert conditions based on the patient standard of care. For example, the caregiver can establish a particular range of bed configurations and define conditions for other parameters associated with the patient support system 28 and the patient care devices 30, for example, that are required by the patient's standard of care. For example, the head elevation of a hospital bed for certain patients recovering from pulmonary distress may be limited to a certain elevation for most of the day. The system 20 can provide monitoring of parameters such as the hospital bed head elevation, and provide an indication of an alert condition when the bed is outside of the specified range, or upon the bed having been outside of the specified range for a predetermined amount of time. Thus, a caregiver at a geographically distant location can select parameters to monitor and alter conditions. The parameters and alter conditions are subsequently transmitted to and stored in memory 48 of portal 46 which is located at a patient's home environment. The processor 47 then operates to determine when the programmed alert conditions are violated and to initiate the transmission of an alert signal to the system 64 through the infrastructure 26. Monitor system 64 then responds with an alert indication or other specified alert response protocol.

Similarly, a patient at risk of or recovering from pressure ulcer development may be required to maintain a certain minimum amount of movement in the bed. The system 20 can notify the caregiver upon the patient having not significantly or sufficiently moved during a predetermined period of time.

The system 20 can also be used to monitor the effectiveness of in-home therapies. For example, the system 20 can be used in conjunction with therapy devices such as a percussion treatment vest and a pulse oximetry device. Using caregiver interface 66, the caregiver can select monitored parameters and alert conditions for these devices that would trigger an indication that the therapy is ineffective.

The patient interface 34 and specifically the caregiver call switch 35 may be used to initiate voice communications with a caregiver in the event the in-home patient becomes distressed or otherwise requires the assistance of the caregiver. Activating the caregiver call switch 35 functions much in the same way in-hospital nurse call devices operate. For example, a display and audio request may be annunciated at the caregiver interface 66 and/or using other caregiver communication devices 76, 79, 81, 83, 85, and 97 until the caregiver responds to and makes contact with the in-home patient.

The above-mentioned and other datalinks shown in FIGS. 1A and 1B, including the communication infrastructure 26, may be any wired or wireless communication connections known in the art, including for example, Ethernet, T1, ELA/TIA-464A compliant E & M or other analog tie lines, TCP/IP, WIMAX IEEE 802.16, WIFI IEEE 802.11, or other wireless or wired connections. Wireless communication connections may be radiofrequency or non-radiofrequency connections, including dedicated channels or network infrastructures.

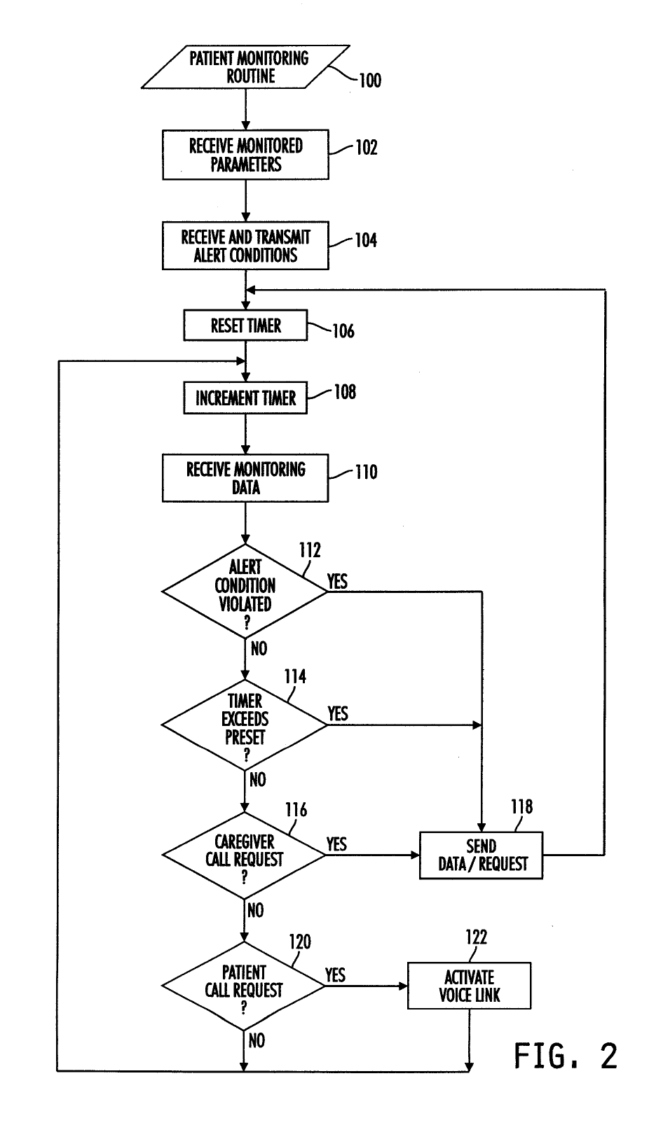

Referring now to FIG. 2, a flow chart is shown of one illustrated embodiment of a software algorithm or routine for patient monitoring in the in-home portion 22 of the caregiver communication system 20. For example, the patient monitoring routine 100 may be executed by one or more of the patient communication portal 46, the device communication portal 52, the transmitter/receiver controller 56, and the patient support system 28 and associated electronic packages. In the illustrative embodiment, the patient monitoring routine 100 is executed by the processor 47 associated with the patient communication portal 46. The routine 100 begins at step 102. At step 102 the portal 46 receives the selection of monitored parameters from the monitored system 64. The monitored parameters can be stored in memory 48 and used to determine which data signals to transmit and the frequency at which to initiate communications and send the data signals to the monitor system 64. Alternatively, a communications channel may be opened for continuous or near-continuous transmission of signals relating to the monitored parameters.

At step 104, the portal 46 receives the predefined alert conditions from the monitor system 64 and, if required, transmits the alert conditions to other in-home devices, for example, to the bed sensor system 32 and the device communication portal 52. The alert conditions relate to predefined limits associated with at least one of the monitored parameters. For example, the alert conditions may be used to trigger a special transmission and indication to the caregiver that monitored parameters have violated a specified condition.

At step 106, the portal 46 resets a timer used to provide periodic transmission of data to the monitor system 64. At step 108, the portal 46 increments the timer. At step 110, the portal 46 receives one or more data signals from the bed sensor system 32, the patient interface 34, and the device communication portal 52. If, at step 112, the portal 46 determines that one or more of the received data signals violate an alert condition, execution of the routine advances to step 118 where the portal 46 transmits data relating to the alert condition to the monitor system 64. After step 118 is executed, execution of the routine 100 returns to step 106.

If the portal 46 determines at step 112 that an alert condition has not been violated, then the routine 100 advances to step 114. If at step 114, the portal 46 determines that the periodic transmission timer exceeds a preset limit, then execution of the routine advances to step 118 in which selected signals are transmitted to the monitor system 64. If, at step 114, the portal 46 determines that the timer is not exceeded, execution of the routine 100 advances to step 116.

If, at step 116, the portal 46 determines that the data signal indicates that the caregiver call switch 35 has been actuated by the patient, execution of the routine 100 advances to step 118 where the portal 46 transmits the call request. Otherwise, execution of the routine 100 advances to step 120. If, at step 120, the portal 46 determines that data has been received from the monitor system 64 indicating that the caregiver has actuated the patient call selection 91, execution of the routine 100 advances to step 122. As step 122, the portal 46 activates data signals to establish a voice communications link between the patient interface 34 and the caregiver interface 66 or another caregiver communication device 76, 79, 81, 83, 85, and 97. After execution of step 122 or if in step 120 it is determined that the patient call selection 91 has not been activated, execution of the routine 100 returns to step 108.

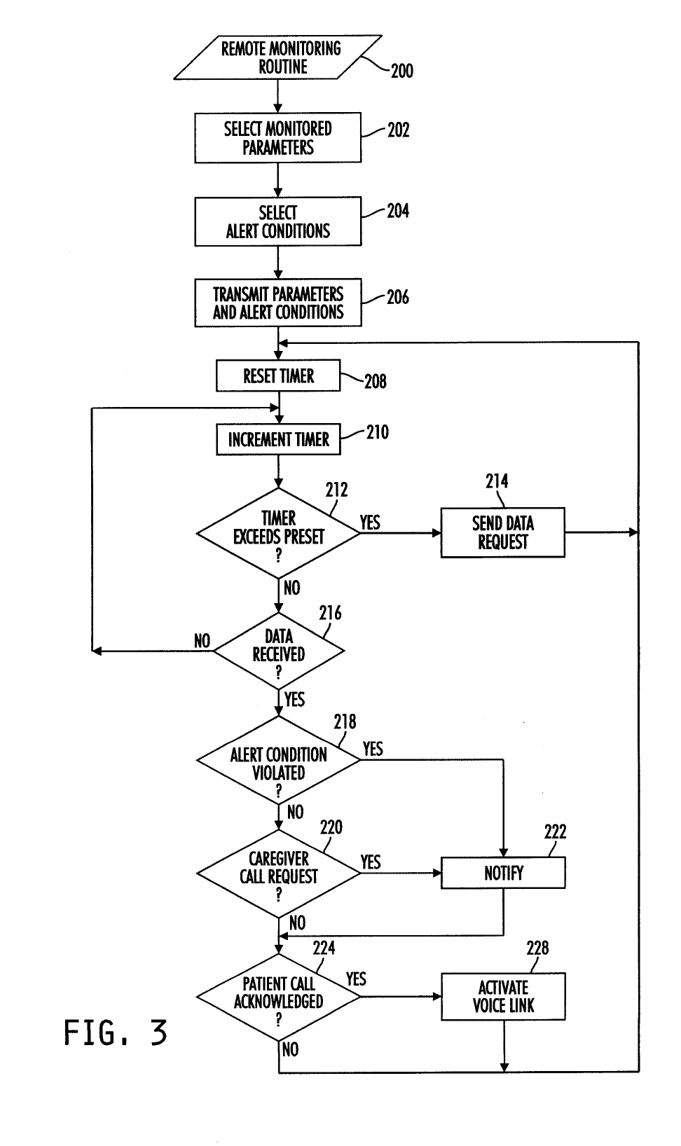

Referring now to FIG. 3, a flow chart is shown of one illustrative embodiment of a software algorithm or routine 200 for providing remote monitoring of the patient and the patient support system 28 and the patient care devices 30. The remote monitoring routine 200 may be executed by the processor 88 of the nurse call monitor system 64 and/or other components in the healthcare facility portion 24 of the caregiver communication system 20.

At step 202, the monitor system 64 receives and stores in the memory 89 the monitoring conditions selected by the caregiver, for example, using the display 90 and a keyboard of the caregiver interface 66. In step 204, the monitor system 64 receives and stores in the memory 89 the alert conditions selected by the caregiver. At step 206, the monitor system 64 transmits the monitored parameters and alert conditions and any associated signals to the patient communication portal 46. Specifically, the monitor communication portal 62 receives the conditions from the monitor system 64 and transmits them over the communication infrastructure 26 to the patient communication portal 46.

At step 208, the monitor system 64 resets the interval timer to zero. The interval time is used to ensure that the monitor system 64 periodically receives new signals from the patient communication portal 46. At step 210, the monitor system 64 increments the interval timer. If, at step 212, the monitor system 64 determines that the interval timer has exceeded a preset interval, execution of the routine 200 continues at step 214, otherwise execution of the routine 200 continues at step 216. At step 214, the monitor system 64 transmits a request for the patient communication portal 46 to send data. After step 214 is completed, the execution of the routine 200 returns to step 208.

If, at step 216 the monitor system 64 determines that new data has been received from the patient communication portal 46, execution of the routine 200 continues at step 218. Otherwise, execution of the routine 200 returns to step 210. If, at step 218, the monitor system 64 determines that received data signals indicate an alert condition has been violated, execution of the routine 200 continues at step 222. Otherwise, execution of the routine continues at step 220. At step 222, the monitor system 64 provides notification and or indication of an alert condition. For example, a text, prerecorded, or other data signal may be transmitted to the caregiver interface 66 and other caregiver communication devices, for example the handset 81, pager 83, badge 85, telephone 79, outside telephone 97, and audio station 76. Additionally at step 222, the monitor system 64 may receive acknowledgement of the notification from the caregiver interface 66 or other caregiver communication devices. If acknowledgement is not received, for example within a preset time duration, then at step 222, the monitor system 64 may also escalate the alert to a second caregiver or other recipient.

If, at step 220, the monitor system 64 determines that a data signal has been received indicating that the caregiver call switch 34 has been actuated, execution of the routine continues at step 222 to notify the caregiver of the call request. Otherwise, execution of the routine 200 continues at step 224. Additionally at step 222, the monitor system 64 may include acknowledgement handling procedures for a call request as discussed above for other notifications.

If, at step 224, the monitor system 64 determines that received data indicates the caregiver has acknowledged a patient call request, execution of the routine continues at step 228. Otherwise, execution of the routine 200 returns to step 208. At step 228, the monitor system 64 provides the data connectivity required for activating a voice communications link between the patient interface 34 and the caregiver interface 66 or one of the other caregiver communication devices, for example, the handset 81, pager 83, badge 85, telephone 79, outside telephone 97, and audio station 76. After execution of step 228, execution of the routine 200 returns to step 208.

While the invention has been illustrated and described in detail in the foregoing drawings and description, the same is to be considered as illustrative and not restrictive in character, it being understood that only illustrative embodiments thereof have been shown and described and that all changes and modifications that come within the sprit of the invention as defined in the following claims are desired to be protected.

1. A patient monitoring system comprising:
a patient support system for a home environment;
a sensor system configured to generate a first signal relating to at least one parameter of the patient support system;
a first communication portal coupled to the sensor system by a first datalink, the first communication portal configured to receive the first signal from the sensor system;
a second communication portal positioned geographically distant from the first communication portal, the second communication portal coupled to the first communication portal by a second datalink and configured to receive the first signal from the first communication portal; and
a monitoring device in communication with the second communication portal by a third datalink, the monitoring device configured to receive and process the first signal from the second communication portal.

2. The patient monitoring system of claim 1, further comprising a third communication portal and a caregiver communication device, the third communication portal coupling the monitoring device and the caregiver communication device, the caregiver communication device configured to receive communications relating to the first signal.

3. The patient monitoring system of claim 1, further comprising a caregiver interface coupled to the monitoring device and configured to provide indications relating to the first signal.

4. The patient monitoring system of claim 3, further comprising a patient interface coupled to the first communication portal, and wherein the patient interface and the caregiver interface are configured to provide voice communication between a patient and caregiver.

5. The patient monitoring system of claim 1, wherein the patient support system includes at least one of a patient support surface and a patient care device.

6. The patient monitoring system of claim 5, further comprising a fourth communication portal coupling the first communication portal and the patient care device and configured to receive a second signal from the patient care device and to transmit the second signal to the first communication device, the second signal relating to at least one parameter of the patient care device.

7. The patient monitoring system of claim 6, wherein:
the first datalink includes at least one communication controller;
the at least one communication controller is coupled between the first communication portal and the fourth communication portal;
the at least one communication controller combines the first signal received from the sensor system and the second signal received from the fourth communication portal for transmission through the first communication portal and the second communication portal to the monitoring device; and
the at least one communication controller separates a plurality of signals received from the first communication portal for transmission to at least one of the sensor system and the fourth communication portal.

8. The patient monitoring system of claim 6, wherein the first communication portal is configured to periodically transmit the first and second signals to the monitoring device.

9. The patient monitoring system of claim 1, wherein:
the first communication portal is configured to transmit a third signal to the monitoring device upon a parameter violating a predefined condition, the parameter being associated with a status of at least one of the sensor system and the patient care device; and
the predefined condition may be transmitted from the monitoring device to the first communication portal.

10. A patient monitoring system for a patient support system, comprising:
a patient support system sensor configured to generate signals relating to a status of the patient support system;
a first communication portal coupled to the patient support system sensor and configured to receive and transmit the signals;
a second communication portal located geographically distant from the first communication portal and configured to be coupled to the first communication portal by a datalink, to receive the signals from the first communication portal, and to transmit the signals; and
a monitoring device in communication with the second communication portal and configured to receive the signals from the second communication portal and to provide an alert to a caregiver upon the signals violating one of a plurality of predefined conditions.

11. The patient monitoring system of claim 10, wherein the patient support system sensor is associated with and monitors a parameter relating to a status of at least one of a patient support surface, a patient care device, a patient monitoring device, an IV pump, a sequential compression device, a pulmonary percussion therapy device, an environmental monitoring device, an oxygen concentrator, patient blood pressure, patient temperature, patient respiration rate, patient blood oxygen saturation, patient heart rate, patient position, patient weight, a performance value for a component of the patient support surface, the position of a component of the patient support surface, a pressure relief component of the patient support surface, and a percussion therapy component of the patient support surface.

12. The patient monitoring system of claim 10, further comprising a caregiver interface coupled to the monitoring device, the caregiver interface configured to allow a caregiver to set the plurality of predefined conditions, and wherein the monitor system is configured to store the plurality of predefined conditions and to provide the alert based on comparing the signals to the plurality of predefined conditions.

13. The patient monitoring system of claim 10, wherein the first communication portal is configured to receive and store the plurality of predefined conditions and further configured to initiate a communication connection through the datalink and the second communication portal to provide an alert signal to the monitoring device upon signals violating at least one of the plurality of predefined conditions.

14. The patient monitoring system of claim 10, wherein at least one of the plurality of predefined conditions relates to a patient Standard of Care.

15. A patient monitoring system for an in-home patient support system, comprising:
a sensor system configured to generate signals relating to at least one parameter of the patient support system;
a first communication portal coupled to the sensor system by a first datalink;
a second communication portal located geographically distant from the first communication portal and coupled to the first communication portal by a second datalink; and
a monitoring device in communication with the second communication portal by a third datalink, the monitoring device configured to store conditions relating to the signals;
wherein the first communication portal, the second communication portal, and the monitoring device are configured to transmit and receive data relating to the signals and the conditions.

16. The patient monitoring system of claim 15, wherein the monitoring device includes a processor and software enabling the monitoring device to:
provide selection of a condition relating to the at least one parameter of the patient support system;
receive the data relating to the signals and the condition;
compare the condition to the received data; and
provide a caregiver alert upon the received data satisfying the condition.

17. The patient monitoring system of claim 15, wherein the first communication portal includes a processor and software enabling the first communication portal to:
receive a condition relating to the at least one parameter of the patient support system;
receive signals relating to the at least one parameter of the patient support system; and
selectively transmit data to the monitoring device based on a function of the condition and the signals.

18. The patient monitoring system of claim 17, wherein the data is transmitted at least periodically.

19. The patient monitoring system of claim 15, wherein the first communication portal includes a second communication portal, the first communication portal is associated with a patient support surface and the second communication portal is associated with a patient care device.

20. The patient monitoring system of claim 15, further comprising:
a patient interface coupled to the first communication portal; and
a caregiver interface coupled to the second communication portal;
wherein the patient interface and caregiver interface are configured to provide voice communication between a patient and a caregiver.
